(19) United States
(12) Patent Application Publication
(10) Pub. No.: US 2022/0295819 A1
(43) Pub. Date: Sep. 22, 2022

(54) METHOD OF MAKING COCOA BUTTER-DERIVED PRODUCTS CONTAINING CANNABINOIDS

(71) Applicant: CANOPY GROWTH CORPORATION, Smiths Falls, CA (US)

(72) Inventors: Ben GEILING, Smiths Falls (CA); Erica GILMOUR, Almonte (CA); Andrew GILMOUR, Almonte (CA); Brandon PASQUARIELLO, Smiths Falls (CA); Matthew COULTER, Smiths Falls (CA)

(21) Appl. No.: 17/612,770

(22) PCT Filed: May 22, 2020
(86) PCT No.: PCT/CA2020/050692

§ 371 Date: Nov. 19, 2021

Related U.S. Application Data
(60) Provisional application No. 62/852,038, filed on May 23, 2019.

(51) Int. Cl. A23G 1/48; A23G 1/36; A23G 1/42; A23D 9/007; A23G 1/00; A23L 33/105; A23P 10/35

(52) U.S. Cl. CPC A23G 1/48; A23G 1/36; A23G 1/42; A23D 9/007; A23G 1/0026; A23L 33/105; A23P 10/35; A23D 7/001

(57) ABSTRACT

The present technology generally relates to methods of making cocoa butter-derived products that comprise cannabinoids. The present technology further generally relates to cocoa butter-derived products resulting from such methods.

### CROSS-REFERENCE TO RELATED APPLICATION

This application claims the benefit of and priority to U.S. provisional patent application No. 62/852,038, filed on May 23, 2019, the content of all of which is herein incorporated in entirety by reference.

### FIELD OF TECHNOLOGY

The present technology generally relates to methods of making cocoa butter-derived products that comprise cannabinoids as well as to cocoa butter-derived products resulting from such methods.

### BACKGROUND INFORMATION

The cannabis plant has many naturally occurring substances that are of great interest in science and medicine. Isolated compounds from the cannabis plant include Δ⁹-tetrahydrocannabinol (THC), cannabidiol (CBD), cannabichromene (CBC), cannabigerol (CBG), cannabidivarin (CBDV), among other compounds. While THC has psychoactive effects, CBD, CBC, CBG, and CBDV do not.

Isolated compounds from the cannabis plant are called cannabinoids. There are at least eighty-five (85) cannabinoids which have been isolated from the cannabis plant.

Plants in the cannabis genus include Cannabis sativa, Cannabis ruderalis, and Cannabis indica. These plants are the natural sources of cannabinoids. Cannabinoids are also available in synthetic forms. Methods to synthesize cannabinoids in lab settings were discovered and are practiced currently. Synthetic cannabinoids are more targeted; meaning the synthetic compound usually comes isolated without other cannabinoids mixed in. Cannabinoids can be isolated by extraction from cannabis plants.

Cannabis products consumption has many benefits. Cannabis products are widely used to increase appetite, induce sleep, prevent nausea, and relieve pain, among other beneficial effects. Cannabinoids contained in these products are responsible for these desirable effects.

Many ways exist to incorporate cannabinoids into the user's daily ingredients however; there is a demand for tasty edible products with cannabinoid content, such as for example coca-derived products. Cocoa containing a desirable amount of cannabinoid provides alertness, represents an important source of healthy ingredients, such as e.g. minerals, vitamins, polyphenols (especially catechins, anthocyanidins and proanthocyanidins), and is palatable to the consumer.

Methods for obtaining cocoa containing a desirable amount of cannabinoids have been proposed. Some of these methods involve mixing raw cannabis or cannabis extract or distillate with an oil and adding the mixture to chocolate. One drawback of these methods is that they do not allow for an optimal integration of the cannabinoids into the cocoa butter-derived products.

As such, there remains a need for methods for adding cannabinoids into cocoa butter-derived products that provide a better integration of the cannabinoids into the cocoa butter and into the cocoa butter-derived product to allow the consumer to experience the full benefit of cannabinoids and of the cocoa butter-derived products.

### Summary of Disclosure

According to various aspects, the present technology relates to a method of making cannabinoid-containing cocoa butter, the method comprising mixing cocoa butter with a cannabinoid concentrate during preparation of the cocoa butter. In some instances, the mixing of the cocoa butter with the cannabinoid concentrate involves infusing the cocoa butter with the cannabinoid concentrate. In some further instances, the mixing is performed before conching of the cocoa butter, or during refining of the cocoa butter, or after grinding of the cocoa butter, or after grinding but before conching of the cocoa butter.

According to various aspects, the present technology relates to a method of making a cocoa butter-derived product comprising cannabinoid, the method comprising: i) mixing cocoa butter with a cannabinoid concentrate to obtain a cannabinoid-containing cocoa butter; and ii) formulating the cannabinoid-containing cocoa butter to the cocoa butter-derived product.

According to various aspects, the present technology also relates to cocoa-derived products made from the cannabinoid-infused cocoa butter as defined herein. In some instances, the cocoa-derived product of the present technology is a chocolate made from the cannabinoid-infused cocoa butter as defined herein.

Other aspects and features of the present disclosure will become apparent to those ordinarily skilled in the art upon review of the following description of specific embodiments.

### DETAILED DISCLOSURE OF EMBODIMENTS

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.25, 1.5, 1.75, 2, 2.45, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. The term “or” as used herein should in general be construed non-exclusively. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

As used herein, the term “comprise” is used in its non-limiting sense to mean that items following the word are included, but items not specifically mentioned are not excluded.

As used herein, the term “Cannabis” refers to the genus of flowering plants in the family Cannabaceae. The expressions “Cannabis sativa” and “C. sativa” are used herein interchangeably.

As used herein, the term “cannabinoid” refers to a chemical compound belonging to a class of secondary compounds commonly found in plants of genus cannabis, but also encompasses synthetic and semi-synthetic cannabinoids. The most notable cannabinoid is tetrahydrocannabinol (THC), the primary psychoactive compound in cannabis. Cannabidiol (CBD) is another cannabinoid that is a major constituent of the phytocannabinoids. There are at least 113 different cannabinoids isolated from cannabis, exhibiting varied effects.

Synthetic cannabinoids and semi-synthetic cannabinoids encompass a variety of distinct chemical classes, for example and without limitation: the classical cannabinoids structurally related to THC, the non-classical cannabinoids (cannabimimetics) including the aminoalkylindoles, 1,5 diarylpyrazoles, quinolines, and arylsulfonamides as well as eicosanoids related to endocannabinoids.

In many cases, a cannabinoid can be identified because its chemical name will include the text string “*cannabi*”. However, there are a number of cannabinoids that do not use this nomenclature.

Within the context of this disclosure, where reference is made to a particular cannabinoid, each of the acid and/or decarboxylated forms are contemplated as both single molecules and mixtures. In addition, salts of cannabinoids are also encompassed, such as salts of cannabinoid carboxylic acids.

As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

As used herein, the expression “cannabinoid concentrate” refers to products made from the cannabis plant that have been processed to keep only the most desirable plant compounds (primarily the cannabinoids), while removing excess plant material and other impurities. As used herein, the expression “cannabinoid concentrate” includes one or more of cannabinoid distillate and cannabinoid isolate (e.g., crystalline CBD).

The expression “cannabis oil” as used herein refers to a mixture of compounds obtained from the extraction of cannabis plants. Such compounds include, but are not limited to, cannabinoids, terpenes, terpenoids, and other compounds found in the cannabis plant. The exact composition of cannabis oil depends on the strain of cannabis that is used for extraction, the efficiency and process of the extraction itself, and on any additives that might be incorporated to alter the palatability or improve administration and/or bioavailability of the cannabis oil.

The term “eluate” as used herein refers to a solution that is collected after contacting a plant material, such as raw cannabis plant material, with an extraction solvent. The eluate can contain dissolved cannabinoids as well as other compounds. The term “filtrate” refers to a solution that has passed through a membrane or strainer of variable porousness or permeability to remove either particulate matter or unwanted compounds. As used herein, the term “distillate” refers to a solution that has been concentrated by any known means of evaporation or distillation. In some embodiments of the present technology, the filtrate is evaporated to form a distillate. The term “extract” as used herein refers to a solution that has been purged or dehydrated to remove residual solvent. In some embodiments of the present technology, the extract is formed by purging or dehydrating the distillate using any known means in the art. As used herein, the term “isolate” refers to a chemical substance that has been separated from foreign or contaminating substances. Pure results of a successful purification process are termed isolate. In some embodiments of the present technology, the isolate is refined distillate.

As used herein, the expression “cocoa butter-derived product” refers to products that are made from cocoa butter, such as, for example, chocolate (e.g., white chocolate, milk chocolate, and dark chocolate). Cocoa butter contains a high proportion of saturated fats as well as monounsaturated oleic acid, which typically occurs in each triglyceride. The predominant triglycerides are palmitic acid, oleic acid, and stearic acid. Cocoa butter typically has a melting point of around 34-38° C.

Without wishing to be bound to any specific theory, embodiments of the present technology have been developed based on the elucidation by the present discoverers that adding a cannabinoid concentrate to cocoa butter allows for an improved integration of the cannabinoid into the cocoa-butter and into the final cocoa butter-derived product made with such cannabinoid-containing cocoa butter.

In one embodiment, the present technology thus relates to a method of making cannabinoid-containing cocoa butter by adding a cannabinoid concentrate to cocoa butter.

i) Processing of Cocoa Beans

Conventional methods for processing cocoa beans into cocoa-derived products such as, for example, chocolate, involve an initial step of cleaning the cocoa beans (i.e., cleaning step) at the farm or centralized facility. In some instances, the cleaning step involves fermenting the beans. Fermentation occurs when the pulp surrounding the cacao bean is converted into alcohol by the yeasts present in the air and the heat generated. The beans are mixed gently during this process to introduce oxygen, which turns the alcohol into lactic and acetic acid. The fermentation process can take up to eight days, depending on the species of cacao bean.

The cocoa beans are then passed through a machine that removes dried cocoa pulp, pieces of pod and other extraneous material. The last vestiges of wood, jute fibres, sand and finest dust are extracted by various pieces of equipment. To bring out the characteristic chocolate aroma, the beans are roasted in large rotary cylinders (i.e., roasting step). Depending upon the variety of the beans and the desired end result, the roasting lasts from between about 30 minutes to about 2 hours at temperatures of about 121° C. or higher. As the beans turn over and over, their moisture content drops, their color changes to a rich brown, and the characteristic aroma of chocolate becomes evident.

The cocoa beans are then cooled quickly and their thin shells or “chaff” which have become brittle by roasting, are removed (i.e., shell removal step). A winnowing machine passes the beans between serrated cones so they are cracked rather than crushed. In the process, a series of mechanical sieves separate the broken pieces into large and small grains (nibs) while fans blow away the thin, light shell (chaff) from the bean or “nibs”. The nibs, which contain about 53% cocoa butter, pass through refining mills and are ground between large grinding stones or heavy steel discs creating a cocoa paste (i.e., nibs grounding step). The cocoa paste is then subjected to hydraulic pressure to give rise to cocoa butter. Heat generated by grinding causes the cocoa butter to melt and to form a fine paste or liquid known as chocolate “liquor”. When the liquid is poured into molds and allowed to solidify, the resulting cakes are unsweetened or bitter chocolate. Up to this point, the manufacturing of cocoa and chocolate is identical. The by-product of cocoa, cocoa butter is the essential component of chocolate.

To make cocoa powder, chocolate liquor is pumped into hydraulic presses weighing up to 25 tons, and when the pressure is applied, 80% cocoa butter is removed (i.e., separation of cocoa from cocoa butter step). The fat drains away through metallic screens as a yellow liquid, and then is collected for use in chocolate manufacturing. The “cake” which is left may eventually be made into cocoa powder by being further crushed, milled and finely sifted. Next the cake is put into ball mills, where many thousands of stainless steel balls reduce the tiny particles of cocoa and sugar down to a size of about 20 microns. Additional ingredients such as, for example, milk, flavors, sugar, may then be added. Milk chocolate is made by adding milk, sugar, cocoa butter and other ingredients to the bitter chocolate liquor. At this point, chocolate is prepared in according to individual recipes. The blending of the various types of cocoa pastes and other ingredients determine the ultimate taste.

Conching machines are then used to knead the chocolate paste (i.e., conching step). This process develops flavors and changes the texture during controlled temperatures. This step allows the separate flavors of the individual ingredients to combine. Conches are equipped with heavy rollers that plow back and forth through the chocolate paste, anywhere from a few hours to several days. Contemporary technologies can grind the chocolate particles extremely fine, which can reduce conching times. Conching time may vary from any where between about 4 hours to 96 hours. Under regulated speeds and temperatures, these rollers can produce different degrees of agitation and aeration to create distinct chocolate flavors. The process can eliminate any remaining bitterness by aerating the chocolate and expelling volatile acids. Additional cocoa butter and lecithin may be added which help to achieve the characteristic velvet smoothness. And as the ultimate homogeneity of the ingredients is developed, a soft film of cocoa butter begins to form around each of the small particles. The chocolate no longer seems sandy, but dissolves meltingly on the tongue. In some manufacturing setups, there is an emulsifying operation that either takes the place of conching (or supplements conching). Emulsifying is breaking up sugar crystals and other particles in the chocolate mixture to give it a fine, velvety smoothness. This thickens the chocolate and imparts the right flow properties for filling the moulds. The still warm conched chocolate is placed in a tempering machine so that it can be slowly and steadily cooled, in a way that promotes harmonious “beta five” crystals that enable shelf-stable, shiny, brittle chocolate.

ii) Cannabinoid-Containing Cocoa Butter

According to one embodiment, the present technology provides a method of preparing cannabinoid-containing cocoa butter. The method includes the step of adding a cannabinoid concentrate to cocoa butter. In some implementations of this embodiment, the cannabinoid concentrate is added to the cocoa butter during the conching step of the cocoa butter preparation. In some implementations of this embodiment, the cannabinoid concentrate is added to the cocoa butter before the conching step of the cocoa butter preparation. In some implementations of this embodiment, the cannabinoid concentrate is added to the cocoa butter during the refining step of the cocoa butter preparation. In some implementations of this embodiment, the cannabinoid concentrate is added to the cocoa butter after the grinding step of the cocoa butter preparation. In some implementations of this embodiment, the cannabinoid concentrate is added to the cocoa butter after the grinding step but before the conching step of the cocoa butter preparation. In some instances, the step of adding the cannabinoid concentrate to the cocoa butter involves infusing the cocoa butter with the cannabinoid concentrate to give rise to cannabinoid-infused cocoa butter.

In some embodiments, the ratio of cannabinoid:cocoa butter depends on the concentration of cannabinoid concentrate. In some instances, the mass ratio of cannabinoid concentrate:cocoa butter is between about 1:99 and about 99:1. In some instances, the mass ratio of cannabinoid concentrate:cocoa butter is between about 5:95 and about 95:5. In some instances, the mass ratio of cannabinoid concentrate:cocoa butter is between about 10:90 and about 90:10. In some instances, the mass ratio of cannabinoid concentrate:cocoa butter is between about 5:95 and about 50:50. In some instances, the mass ratio of cannabinoid concentrate:cocoa butter is between about 5:95 to about 25:75. In some instances, the mass ratio of cannabinoid concentrate:cocoa butter is about 15:85. In some instances, the mass ratio of cannabinoid concentrate:cocoa butter is about 20:80. In some instances, the mass ratio of cannabinoid concentrate:cocoa butter is about 25:75. In some instances, the mass ratio of cannabinoid concentrate:cocoa butter is about 33:67. In some instances, the mass ratio of cannabinoid concentrate:cocoa butter is between about and 50:50 and about 95:5. In some instances, the mass ratio of cannabinoid concentrate:cocoa butter is between about 75:25 and about 95:5. In some instances, the mass ratio of cannabinoid concentrate:cocoa butter is about 85:15. In some instances, the mass ratio of cannabinoid concentrate:cocoa butter is about 80:20. In some instances, the mass ratio of cannabinoid concentrate:cocoa butter is about 75:25. In some instances, the ratio of cannabinoid concentrate:cocoa butter is about 67:33. Certain ratios of cannabinoid concentrate:cocoa butter allow for the cocoa butter to be solid at room temperature for handling purposes. Examples of mass ratios include about 20:80 cannabinoid concentrate:cocoa butter and about 15:85 cannabinoid concentrate:cocoa.

In some instances, the mass ratio of cannabinoid concentrate:cocoa butter is between about 10:90 and about 50:50. In some instances, the mass ratio of cannabinoid concentrate:cocoa butter is between about 10:90 and about 33:67. In some instances, the mass ratio of cannabinoid concentrate:cocoa butter is between about 10:90 and about 20:80. In some instances, the mass ratio of cannabinoid concentrate:cocoa butter is between about 20:80 and about 50:50. In some instances, the mass ratio of cannabinoid concentrate:cocoa butter is between about 20:80 and about 33:67. In some instances, the mass ratio of cannabinoid concentrate:cocoa butter is between about 33:67 and about 50:50.

In some instances, the mass ratio of cannabinoid concentrate:cocoa butter is between about 50:50 and about 90:10. In some instances, the mass ratio of cannabinoid concentrate:cocoa butter is between about 67:33 and about 90:10. In some instances, the mass ratio of cannabinoid concentrate:cocoa butter is between about 80:20 and about 90:10. In some instances, the mass ratio of cannabinoid concentrate:cocoa butter is between about 50:50 and about 80:20. In some instances, the mass ratio of cannabinoid concentrate:cocoa butter is between about 67:33 and about 80:20. In some instances, the mass ratio of cannabinoid concentrate:cocoa butter is between about 50:50 and about 67:33.

In some instances, the mass ratio of cannabinoid concentrate:cocoa butter is between about 15:85 and about 85:15. In some instances, the mass ratio of cannabinoid concentrate:cocoa butter is between about 20:80 and about 80:20. In some instances, the mass ratio of cannabinoid concentrate:cocoa butter is between about 25:75 and about 75:25. In some instances, the mass ratio of cannabinoid concentrate:cocoa butter is between about 33:67 and about 67:33. In some instances, the mass ratio of cannabinoid concentrate:cocoa butter is about 50:50.

In some embodiments, the cannabinoid concentrate is added to cocoa butter at an infusion temperature ranging from between about 30° C. and about 90° C., or between about 50° C. and about 90° C., or between about 70° C. and about 90° C., or between about 40° C. and about 80° C., or between about 60° C. and about 80° C., or between about 40° C. and about 60° C. In some embodiments, the cannabinoid concentrate is added to cocoa butter at an infusion temperature ranging from between about 30° C. and about 45° C., or between about 35° C. and about 40° C., or between about 40° C. and about 45° C.; or at a temperature of about 40° C., about 45° C., about 50° C., about 55° C., about 60° C., about 65° C., about 70° C., about 75° C., about 80° C., about 85° C., or about 90° C.

In some embodiments, the step of adding the cannabinoid concentrate to the cocoa butter is performed over mild heat to promote the formation of a homogenous mixture of cannabinoid concentrate and cocoa butter. In some instances, mixing or infusing the cocoa butter with the cannabinoid concentrate is carried out for a period of at least about 30 minutes, or at least about 40 minutes, or at least about 50 minutes, or at least about 60 minutes, or at least about 90 minutes or at least about 120 minutes. In some other instances, mixing or infusing the cocoa butter with the cannabinoid concentrate is carried out until all the cannabis concentrate is effectively dissolved and/or distributed in the cocoa butter.

In some embodiments, once the infused cocoa butter is cooled down, the cannabinoids are distributed throughout the cocoa butter. In some instances, the cannabinoids are uniformly distributed throughout the cocoa butter. In some other instances, the cannabinoids are non-uniformly distributed throughout the cocoa butter.

In some embodiments, the addition of cannabinoids to cocoa butter affects the properties of cocoa butter by decreasing the melting temperature, increasing the softness or both. In some embodiments, the cannabinoid-containing cocoa butter of the present technology may be used to prepare cocoa butter-derived products such as, for example, chocolate. To prepare chocolate comprising the cannabinoid-containing cocoa butter of the present technology, the cannabinoid-containing cocoa butter may be added during the tempering stage of the chocolate preparation. In some instance, the cannabinoid-containing cocoa butter may be mixed with non-cannabinoid-infused cocoa butter so as to achieve a required concentration of cannabinoids into the chocolate.

ii) Cannabinoid Concentrate

In some embodiments, the cannabinoid concentrate suitable for addition into the cocoa butter is a cannabinoid isolate. In some instances, the cannabinoid isolate is a CBD isolate. In some instances, the CBD isolate is substantially pure from other materials and contaminants.

As used herein, the term “purified” or “pure” means extracted, isolated, and/or separated from other compounds, formulations, compositions, matter, and/or mass resulting in a greater than 60% purity.

In some embodiments a “purified” cannabinoid (or “purified” terpene) is greater than about 70% pure, greater than about 75% pure, greater than about 80% pure, greater than about 85% pure, greater than about 90% pure, greater than about 91% pure, greater than about 92% pure, greater than about 93% pure, greater than about 94% pure, greater than about 95% pure, greater than about 96% pure, greater than about 97% pure, greater than about 98% pure, or greater than about 99% pure. Within the context of the present disclosure, where a compound comprises stereogenic centers, the term “purified” includes enantiomerically pure compositions and also mixtures of enantiomers or isomers.

Also within the context of the present disclosure, purified compounds may be purposely formulated with other compounds at various levels of purity. Provided that the ingredients used for purposeful formulation are purified prior to the said purposeful formulation, the act of subsequently formulating them does render them not “purified” within the context of an ingredient list.

In an embodiment, the term “purified” may refer to a cannabinoid that is separated from plant matter from which it was derived.

In an embodiment, the term “purified” may refer to a terpene that is separated from plant matter from which it was derived.

In some instances, the CBD isolate has a purity of at least about 90%, at least about 91%, at least about 92%, at least about 93%, at least about 94%, at least about 95%, at least about 96%, at least about 97%, at least about 98%, or at least about 99%. In some instances, the CBD isolate has a purity of 99.5%.

The CBD isolate may be obtained from CBD-dominant and THC-dominant strains. In some instances, the cannabinoid concentrate is crystalized CBD.

In some embodiments, the cannabinoid concentrate suitable for addition into the cocoa butter is a cannabinoid distillate. In some instances, the cannabinoid distillate is a THC distillate. The THC distillate may be obtained from THC-dominant strains. In some instances, the THC distillate may comprise between about 60% and about 90% THC and between about 40% and about 10% other components (e.g., other cannabinoids, fatty acids, waxes or the like). In some instances, the cannabinoid distillate may comprise a THC:CBD ratio of 4:3.

In some instances, the cannabinoid concentrate comprises a mixture of THC distillate and CBD isolate. For example, the cannabinoid concentrate may comprise a THC:CBD ratio of 1:1 prepared by mixing 60% THC distillate and 40% CBD isolate.

In one embodiment, the cannabinoid concentrate which is used in the methods of the present technology is obtained through separation of the cannabinoids from other Cannabis plant materials. The cannabinoid concentrate which results from such separation is an edible cannabinoid concentrate. In some implementations, the cannabinoid concentrate comprises cannabinoid distillate which is obtained by eluting cannabinoids from Cannabis plant materials with a solvent to produce an eluate, filtering the eluate with a filter to produce a filtrate, and evaporating the solvent from the filtrate with a distiller to produce a distillate. In some implementations, the cannabinoid concentrate comprises cannabinoid isolate, which is obtained by eluting cannabinoids from Cannabis plant materials with a solvent to produce an eluate, filtering the eluate with a filter to produce a filtrate, evaporating the solvent from the filtrate with a distiller to produce a distillate, and refining the distillate to obtain the isolate.

In some embodiments, the Cannabis plant material can be plant material from Cannabis indica. In some embodiments, the cannabis plant material can be plant material from Cannabis sativa. In some embodiments, the cannabis plant material can be plant material from a hybrid Cannabis plant such as a hybrid between Cannabis indica and Cannabis sativa. The cannabis plant material can include flowers, buds, trichomes, leaves, stems, portions therein or combinations thereof. In instances where the cannabis plant material is cannabis buds, the buds can be whole buds or buds that are cut or broken into pieces.

The cannabis plant material as well as the extraction solvent are cooled or are frozen prior to the elution step. One of skill in the art will appreciate that the temperature at which the cannabis plant material and the solvent are cooled or frozen as well as the duration of such cooling or freezing depend in part on factors such as the targeted freezing/cooling temperature and the quantity of materials used in the method, as well as the particular extraction solvent and cannabis strain. The solvent can be a predominantly polar solvent. In one embodiment, the solvent can be an alcohol such as, but not limited to, ethanol. The solvent can also be a polar solvent derived from organic sources. In other embodiments, the solvent can include organic ethers, esters, and/or ketones.

Prior to being cooled or frozen, the cannabis plant material may be soaked in the solvent, at or below room temperature, for about 1 to about 2 hours. In some embodiments, the plant material is left to soak without agitation. In one embodiment, the cannabis plant material can also be macerated while soaking in the solvent. The cannabis plant material can be macerated by agitating the cannabis plant material through mechanical or manual force such as by stirring the solvent. The plant material can also be broken apart or ground into finer-sized particles. The extraction solvent can be soaked with the plant material before straining or the extraction solvent can be kept separate before straining. In instances where cannabis plant is soaked/macerated with extraction solvent, incubation time can range from less than about 1 minute to more than about 10 hours. For example, incubation time ranges from less than 1 minute to about 10 minutes, from about 10 minutes to about 30 minutes, from about 30 minutes to about 2 hours, from about 2 hours to about 4 hours, from about 4 hours to about 7 hours, or from about 7 hours to about 10 or more hours.

The solvent is then used to elute cannabinoids, such as THC and CBD, from the cannabis plant material to produce an eluate. In some instances, the elution includes placing the cannabis plant material in a strainer or perforated filter funnel over a collection receptacle and pouring the solvent over the cannabis plant material. The eluate may be collected in the collection receptacle. Any amount of solvent suitable for extracting cannabinoids and other desired compounds can be used. In one or more embodiments, the eluate collected from this step can be poured over the same cannabis plant material again to elute more of the cannabinoids from the cannabis plant material. This elution step can be repeated until the cannabis plant material has been poured over a total of three to six times, or until the coloration of the eluate exhibits hues of green due to accumulation of chlorophyll or other undesired plant material in the eluate. In some embodiments, multiple elution steps are achieved by reusing the collected eluate of the initial pouring step. In some instances, multiple elution steps are achieved by using fresh extraction solvent. In some instances, the volume of extraction solvent is altered in different pouring steps. Typically, the number of pouring steps is terminated before the eluate turns green, which color can indicate an undesirable level of chlorophyll or other undesired plant material accumulation in the eluate. At this point, the eluate produced by the repeated pours can be filtered to yield a final eluate. The final eluate can be filtered using, for example, a mesh filter.

The eluate can be collected in a glass or other container having a lid or other closing mechanism. In some embodiments, the final eluate is further subjected to solarization. Solarization is a process that includes exposing the cannabis extract to a light source to degrade any chlorophyll that has collected with the cannabinoids. The solarization process can be carried out for any amount of time suitable for degrading, or otherwise reducing, the chlorophyll in the extract. Typically, the incubation time will range from fewer than about 5 minutes to more than about 12 hours. The solarization time can depend on factors including, but not limited to, the strength of the light source used. The solarization time can be from about 5 minutes to about 30 minutes, or from about 30 minutes to about 2 hours, or from about 2 hours to about 5 hours, or from about 5 hours to about 12 hours or more. The solarization time can also depend on the desired finished product. In some embodiments, solarization is carried out for about 2 hours. In some embodiments, solarization is carried out for about 10 hours. In some embodiments, solarization is carried out until the extract changes from a nettle green color to a yellow-brown color. In some embodiments, solarization is carried out until the optical density difference (ODD) of the solution reaches a value indicating acceptable chlorophyll levels in the cannabis extract, as measured on a UV-vis spectrophotometer measuring the difference in absorption between wavelengths around 650 nm (red) and around 940 nm (infrared). The measurement of the ODD between these two wavelengths can be used to determine the chlorophyll content in the cannabis extract. One of skill in the art will recognize that there are other techniques available to determine the amount of chlorophyll remaining in extracts.

In one embodiment, the method can include solarizing the eluate which involves exposing the eluate to direct sunlight. In some instances, the eluate can be placed in direct sunlight for at least two hours. In other instances, a plasma light emitter can be used to direct light at the eluate at a light intensity between about 500 to about 2000 photosynthetic photon flux (PPF) for approximately 8 to 10 hours. Solarization can be accomplished using any source of light suitable for degrading chlorophyll. The light source can be, for example, the sun. Another source of light used can be non-natural light sources. Non-natural light sources can include those that emit a full light spectrum in an attempt to mimic natural light, or those that only provide specific wavelengths. Non-natural light sources can also include those that vary spectral outputs and temperatures as time passes, or those that keep a constant spectral output and temperature. In some embodiments, the light source is sunlight. In some embodiments, the light source is a plasma light. The solarization step can be conducted at any temperature suitable for degrading, or otherwise reducing, the chlorophyll in the extract. Solarization of the eluate can cease when the color of the eluate no longer exhibits a green hue or turns from a green color to a yellowish-brown color. In one embodiment, the level of cannabinoids of the eluate is assayed using, for example, high-performance liquid chromatography (HPLC) and ultraviolet (UV) detectors. In some implementations, after the solarization step, the eluate is cooled to temperatures below ambient temperature (i.e., below about 25° C.).

The eluate is then filtered to produce a filtrate using, for example, vacuum filtration. The filtrate can be collected from the vacuum or side-arm flask and undergo evaporation to produce a distillate (i.e., cannabinoid distillate). The filtrate can be distilled using a distiller or an evaporator (e.g., rotary evaporator). The filtrate can be distilled by separating the solvent from the remainder of the filtrate through a selective evaporation and condensation procedure. The filtrate can be distilled or evaporated for any length of time, depending on the desired concentration of distillate. For example, the filtrate can be distilled or evaporated for anytime ranging from about 30 minutes to about 10 hours or more. A person skilled in the art will recognize that depending on the exact method and machinery used, the exact evaporation time required will vary. The filtrate may be evaporated for time intervals ranging from about 30 minutes to about 2 hours, from about 2 hours to about 4 hours, from about 4 hours to about 6 hours, from about 6 hours to about 8 hours, or from about 8 hours to about 10 hours.

After evaporating the solvent from the filtrate, the distillate may optionally be heated above room temperature under controlled conditions for an additional period of time. In some embodiments, the distillate is heated at a controlled temperature for a period of time sufficient to convert acidic cannabinoids to neutral cannabinoids via decarboxylation. The distillate, after evaporation and optional heating, is transferred to a heating flask. A condenser with recirculating chilling fluid is attached on top of the heating flask to condense oil vapors during the heating process.

After distillation and optional heating, the distillate may be filtered through for example, a solid-phase filter medium. Examples of suitable solid-phase filter media include, but are not limited to, silica gel, activated charcoal, activated carbon, diatomaceous earth (Celite), and ion-exchange resins. The distillate can be homogenized or otherwise combined with a suitable solvent prior to the optional filtration step. The homogenized distillate can then be added to a portion of silica gel that has been conditioned (pre-run) in a suitable filter apparatus with the same solvent as added to the distillate. Once the homogenized distillate is fully absorbed on the silica, additional solvent can be added on top of the settled silica. During the silica gel filtration step, the homogenized distillate and added solvent can be pulled through the filter apparatus using a light vacuum or pushed through the filter apparatus using positive pressure applied from above. Alternatively, the homogenized distillate can proceed through the apparatus via gravity filtration. The filtrate can be collected in an appropriate flask prior to removal of solvent via evaporation, as described above. The solvent used in homogenizing the distillate can be any of the solvents discussed above, including ethanol, ethyl acetate, or heptane.

Silica gel can be added to the homogenized distillate in any amount suitable for removing unwanted components via filtration. Silica gel can be added, for example, in an amount ranging from about 1 g of added silica for every 1 g of homogenized distillate (1:1) to about 3 g of added silica for every 1 g of homogenized distillate (3:1). The amount of added silica added to homogenized distillate can range from about 1:1 to about 2:1, or from about 2:1 to about 3:1. In some embodiments the ratio of added silica to homogenized distillate is about 2:1. Additional silica gel is used as the pad or be in the filtration step. Typically, the additional silica gel is used in amounts ranging from about 3 g silica for every 1 g of homogenized distillate (3:1) to about 9:1. For example, the ratio of additional silica to homogenized distillate can range from about 3:1 to about 4:1, from about 4:1 to about 5:1, from about 5:1 to about 6:1, from about 6:1 to about 7:1, from about 7:1 to about 8:1, or form about 8:1 to about 9:1. In some embodiments, the ratio of additional silica to distillate is about 6:1. In some embodiments, the ratio of additional silica to distillate is about 4:1. In some embodiments, the additional silica is loaded into the funnel alone. In some embodiments, the additional silica gel is loaded into the funnel with the same solvent used to homogenize the distillate.

The method can further include dehydrating or purging the distillate (after optional filtration and heating) to further remove any further traces of the solvent. In doing so, the dehydration produces an extract. Dehydration can be achieved using any known means in the art including the use of a food dehydrator, evaporator, or vacuum pump. In some embodiments, the distillate is placed in an open container. In some embodiments, the distillate is place in a sealed container where air pressure can be lowered. In general, purging/dehydration is conducted under conditions sufficient to remove residual solvent from the cannabis oil extract. Residual solvent refers to any solvent (e.g., ethanol) used during the extraction process that remains in the extract after the elution, solarization, filtration, and evaporation steps. The removal of residual solvent can be monitored, for example, by conducting the purge/dehydration step until the weight of the extract stops decreasing (indicating that all volatile solvent has been removed). In some embodiments, removing residual solvent refers to removing at least 90% of the ethanol used in the extraction process from the cannabis oil extract.

In some embodiments, removing residual solvent refers to removing at least 95% of the ethanol used in the extraction process from the cannabis oil extract. In some embodiments, removing residual solvent refers to removing at least 99% of the ethanol used in the extraction process from the cannabis oil extract. Dehydration of residual solvent can be achieved with vacuum pumps providing reduced pressure levels ranging from about 1 mbar to about 500 mbar. In some instances, solvent purging is carried about from about 1 mbar to about 10 mbar, or from about 10 mbar to about 20 mbar, or from about 20 mbar to about 50 mbar, or from about 50 mbar to about 100 mbar, or from about 100 mbar to about 200 mbar, or from about 200 mbar to about 500 mbar.

During the purge/dehydration step, the distillate may be optionally heated to increase the efficiency of the solvent purge. The temperature used for purging/dehydration can be any temperature at or above ambient conditions. For example, heating during the purge/dehydration step can range from about 20° C. to about 200° C. or more. A person of skill in the art will recognize that the time of dehydration required to remove the remaining solvent will depend on the pressure and temperature of the purge/dehydration step as well as the solvent that is being removed. After obtaining the isolate, the composition of the extract can be determined by a variety of the methods. For example, a portion of the isolate can be analyzed by methods including, but not limited to, liquid chromatography/mass spectrometry (LC-MS), gas chromatography/mass spectrometry (GC-MS), and proton nuclear magnetic resonance spectroscopy H-NMR).

A person skilled in the art will appreciate that other methods may be used to obtain cannabinoid concentrate without departing from the present technology.

In some other embodiments, the cannabinoid concentrate that may be used in the methods of the present technology may be obtained from synthetic methods or from biosynthetic methods.

iii) Cannabinoid Concentrate Formulations

Cannabinoids are nearly insoluble in water but soluble in lipids, alcohols and other non-polar organic solvents. Their poor solubility and low dissolution rate in the aqueous gastrointestinal fluids and significant first-pass liver metabolism result in low oral cannabinoid bioavailability. Dissolution rate is a function of the surface area of the particles and solubility. The surface area can be determined through the control of the particle size. Therefore, the bioavailability of cannabinoids can be improved by reduction in their particle size that increases surface area and therefore by integrating them into a delivery system prior to adding the cannabinoid concentrate to the cocoa butter. Examples of delivery systems, include, but are not limited to: capsules (encapsulation), micelles, liposomes, microparticles, nanoparticles, or the like.

As such, in some instances it may be advantageous to formulate the cannabinoid concentrate of the present technology into a delivery system. The resulting delivery system comprising the cannabinoid concentrate may then be added into the cocoa butter as discussed herein. Such formulation of the cannabinoid concentrate may improve the overall bioavailability of the cannabinoids upon oral administration of the cocoa butter-derived product comprising cannabinoids.

In some implementations of these embodiments, the cannabinoid concentrate may be encapsulated. As used herein, the term “encapsulation” refers to the coating of a substance or of a plurality of substances within another material at sizes on the micro or the nano scale. The encapsulated material is referred to as the external phase (also referred to as shell), whereas the core material is referred to as the fill. In some implementations of these embodiments, the cannabinoid concentrate is the internal core of the encapsulation system.

In some further implementations, the cannabinoids are encapsulated by at least one molecular encapsulation agent. Molecular encapsulation can improve the bioavailability and promote the rapid onset of cannabinoids of the present technology. Molecular encapsulation can also provide taste masking effects, so as to reduce or eliminate unpleasant or undesirable tastes in the formulations and final products of the present technology. Non-limiting examples of molecular encapsulation agents include cyclic oligosaccharides such as cyclodextrins including α-, β- and γ-cyclodextrin derivatives and salts thereof, dendrimers, calixarenes, and other molecules and systems capable of forming host-guest complexes, including inclusion complexes, with the cannabinoids of the present technology.

In some implementations, the cannabinoid concentrate of the present technology may be formulated into nanoparticles, such as phospholipid/lipid nanoparticles. Lipid nanoparticles are known for their high degree of biocompatibility, controlled release, efficient targeting, stability, natural biodegradability and high therapeutic index to their payload. Lipid nanoparticles may be assembled as solid lipid nanoparticles, nanostructured lipid carriers and nanospheres.

The phospholipids used for synthesizing the phospholipid nanoparticle may include, but are not limited to: phosphatidycholine, phosphatidylethanolamine, phosphatidylglycerol, phosphatidylserine, phosphatidylinositol, cardiolipin, and the derivatives of these phospholipids. The phospholipids used for synthesizing the phospholipid nanoparticle may further include fatty acids, triglycerides triacylglycerols, acylglycerols, fats, waxes, cholesterol, sphingolipids, glycerides, sterides, cerides, glycolipids, sulfolipids, lipoproteins, chylomicrons and derivatives of these phospholipids.

In some implementations, the cannabinoid concentrate of the present technology may be formulated into nanoemulsions which are carrier systems in the nanometer size comprising a continuous aqueous phase and at least one dispersed oily phase, in which the oily phase comprises at least one amphiphilic lipid such as phospholipids and at least one solubilizing lipid with a monolayer around an amorphous core.

In some further implementations, the cannabinoid concentrate may be incorporated into self-emulsifying drug delivery systems (SEDDS). SEDDS formulations can improve the bioavailability and promote the rapid onset of cannabinoids of the present technology. In some instances, the SEDDS of the present technology are composed of a cannabinoid or cannabis resin, oil phase, surfactant, and in some cases a co-surfactant. Emulsions can be produced using a variety of low energy methods. Low energy methods include, but are not limited to, self-emulsification (direct mixing), slightly elevated temperature, and phase inversion. Self-emulsifying drug delivery systems are liquid at room temperature and typically use high concentrations of surfactant. Surfactant molecules have a hydrophobic tail with a hydrophilic head. This orientation of the surfactant molecules is responsible for the characteristics of the emulsion. When a micellar solution or microemulsion is formed, the surfactant molecules are aligned with their hydrophobic tails toward the center of the particle, and the hydrophilic heads form a layer around the outside of the particle. Having the hydrophilic heads oriented outward allows the particles to stay suspended in aqueous media without separation.

In some instances, the SEDD system of the present technology comprises a second component which is an oil phase or carrier oil, the carrier oil must be easily emulsified and able to dissolve the lipophilic drug. Carrier oils can impart other properties to the SEDDS formulation by further increasing drug uptake, mitigating food effects or promoting chylomicron formation. The formation of chylomicrons aids in the digestion of lipids, they are responsible for the transport of dietary lipids from the intestine to other locations throughout the body. Edible oils that could be used in self-emulsifying drug delivery systems of the present technology include but are not limited to vegetable oils (sunflower, canola, corn, coconut, etc.), MCT oil, Maisine CC, Peceol, Labrafact, Capryol 90, Labrasol and Plurol Oleique.

Surfactants that could be used in a self-emulsifying formulation of the present technology include but are not limited to: polysorbates (Tween), sorbatan esters (Spans), lecithins (phospholipids), sucrose monoesters, TPGS, rhamnolipids and Quillaja saponins.

In some implementations, the cannabinoid concentrate of the present technology may be formulated into solid lipid nanoparticles (SLN). SLN are colloidal drug carriers and dynamic structures that are typically synthesized from phospholipids, lipids, and excipients. They are composed of an outer phase membrane of lipids and/or phospholipids and an inner phase solid lipid inner core. SLN have a mean particle size in the nanometer range. SLN combine the advantages of emulsions, liposomes and polymeric nanoparticles. The solid matrix can protect incorporated cannabinoids against chemical degradation and provide the highest flexibilities in the modulation of the cannabinoid release profiles.

In some implementations, the cannabinoid concentrate of the present technology may be formulated into nanostructured lipid carriers (NLC). NLC are colloidal carriers and a second generation evolvement of SLN. NLC are characterized by an outer phase phospholipid and/or lipid membrane and an inner phase lipid core consisting of a mixture of solid and liquid lipids. NLC have a mean particle size in the nanometer range. NLC are composed of a lipid matrix of cannabinoids with a nanostructure that improves cannabinoid loading and firmly retains the cannabinoids during storage.

In some implementations, the cannabinoid concentrate of the present technology may be formulated into nanospheres (NS). NS are dynamically structured highly stable lipid nanoparticles in the form of nanosized viscoelastic gels. NS are synthesized from biocompatible, and biodegradable essential phospholipids, lipids, and excipients in a unified sequential process. Nanospheres of the present technology have an outer phospholipid membrane and a lipid gel core comprising cannabinoids.

In some further implementations, the cannabinoid concentrate of the present technology may be formulated with coacervates. Coacervates are dynamic structures generated by the association and phase separation of polymeric precursors into polymer rich phase (coacervate) and poor phases. Coacervates can coat or encapsulate oil phases via deposition onto their surfaces. As such, coacervate compositions of the present technology may have a shell or coating comprising adsorbed polymer and an inner core comprising the cannabinoid concentrate. Non-limiting examples of polymeric materials that can be used to form coacervates include one or more of each of: polyelectrolytes, proteins, polypeptides, and polysaccharides. In some further implementations, polymeric materials that can be used to form coacervates include gelatin, Myofibrillar protein, alginate, chitosan, gum Arabic, whey protein, heparin, polycationic peptides, xanthan gum, elastin like peptides, lysozyme, pectin including low methoxyl and high methoxyl pectin, starch, Beta-lactoglobulin, albumin including bovine serum albumin (BSA), polylysine, polyarginine, carboxymethylcellulose, carrageenan, oligosaccharides, casein, and derivatives, salts and combinations thereof. In some instances, the inner core may further comprise at least one oil. Non-limiting examples of oils include any of the oils or carrier oils disclosed anywhere in the specification, and combinations thereof. In some further instances, the shell is crosslinked. Formulation of cannabinoid concentrates with coacervates can provide taste masking effects, so as to reduce or eliminate unpleasant or undesirable tastes in the formulations and final products of the present technology.

In some further implementations, the cannabinoid concentrate of the present technology may be formulated with cubosomes. Cubosomes are dispersions of nano-structured, self-assembled particles of the bicontinuous cubic liquid crystalline phase capable of encapsulating and delivering active ingredients (Spicer, 2005, Karami, 2016, incorporated herein by reference). Cubosomes can be prepared from amphiphilic compounds, including amphiphilic lipids such as glycerides. Examples of lipids that can be used to prepare cubosomes include but are not limited to monoglycerides such as monolaurin, and glycerol esters of and mono-, di- and polyunsaturated fatty acids such as glyceryl monooleate (Peceol, monoolein) and 1-monolinolein. Other ingredients that can be used to prepare cubosomes include phytantriol (3,7,11,15-Tetramethylhexadecane-1,2,3-triol, PHYT) and fatty acids such as lauric and oleic acids. In some embodiments of the current technology, the cubosomes further comprise a nonionic emulsifier, non-limiting examples of which are sorbitan esters such as Polyoxyethylene (20) sorbitan monooleate (Polysorbate 80). The cubosomes can also include a stabilizing agent, surfactant or polymer. Examples of stabilizing agents include but are not limited to β-Casein and CITREM (citric acid esters of monoglycerides and diglycerides). In some embodiments, the cubosomes are prepared in an aqueous system. In some embodiments the cubosomes are dispersed in an aqueous system. In some instances, formulation with cubosomes imparts rapid onset or enhanced bioavailability properties to the cannabinoids of the present technology.

In some further implementations, the cannabinoid concentrate of the present technology may be formulated into organogels. Organogels are structured semi-solid systems that can absorb and immobilize organic liquids, such as cannabinoids, in a three-dimensional network composed of cross-linked, self-assembled gelator fibers. They are thermodynamically stable and exhibit physical properties of a solid. The function and formation of an organogel is attributed to the combination of ingredients and production method. The ingredients required for the formation of an organogel include but are not limited to: i) Solvent, which may be incorporated into organogel formulations to solubilize the organic liquid before solidification in the crosslinked network; ii) Organogelator, which may be is incorporated into the organogel to create the three-dimensional gel network structure (wherein i) and ii) can undergo physical and chemical changes to form self-assembling fibrous structures); and iii) Adjuvants, such as salts or surfactants, which may aid in the optimization of organogel formulations. The adjuvants may affect the morphology of the organogel fibrous network and stability over time. Various types of organogel can be created depending on the solvent/organogelator combination that is used and the desired function of the finished product. In some instances, these include but are not limited to: Pluronic-Lecithin Organogel (PLO); Sorbitan Monostearate (SMO) derived organogels; and 12-hydroxystearic acid (12-HSA) organogels.

In some implementations, the delivery system of the present technology is a water soluble formulation. An example of a water soluble formulation that may be used to deliver the cannabinoid concentrate is discussed in WO 2019104442, incorporated herein by reference. In some cases, the compositions impart enhanced bioavailability or rapid onset properties to the cannabinoids of the present technology. In some cases, the compositions impart taste masking effects to formulations and final products of the present technology, so as to reduce or eliminate undesirable tastes.

In some other implementations, the delivery system of the present technology is an inulin/pectin formulation such as discussed in International Application No. PCT/CA2019/051704, incorporated herein by reference. In some embodiments, these compositions impart taste masking properties to the formulations of the current technology.

In some other implementations, the delivery system of the present technology is a hemp protein formulation such as discussed in WO 2019/213757, incorporated herein by reference. In some embodiments, these formulations impart taste masking properties to the formulations.

In some embodiments, formulations of cannabinoid concentrates of the current technology are prepared via spray drying. In some instances, spray dried formulations comprise microcapsules. In some cases, spray dried formulations comprise encapsulated cannabinoids. In some implementations, the spray dried formulation comprises a delivery system discussed in WO 2019/213757, incorporated herein by reference. In some further implementations, examples of spray dried formulations that may be used to deliver the cannabinoid concentrate are formulations discussed in WO 2019104442 or International Application No. PCT/CA2019/051704, incorporated herein by reference. In some embodiments, spray dried formulations provide taste masking effects, so as to reduce or eliminate undesirable tastes in the formulations and final products of the current technology.

In some cases, the cannabinoid concentrates of the current technology can be formulated into combinations of two or more of the systems described above. For example, a SEDDS formulation can be incorporated into a coacervate system.

In some further implementations, the cannabinoid concentrate of the present invention is lyophilized prior to being added to the cocoa butter. Lyophilization, also known as freeze-drying, is a process whereby water is sublimed from a composition after it is frozen. The frozen solution is then typically subjected to a primary drying step in which the temperature is gradually raised under vacuum in a drying chamber to remove most of the water, and then to a secondary drying step typically at a higher temperature than employed in the primary drying step to remove the residual moisture in the lyophilized composition. The lyophilized composition is then appropriately sealed and stored for later use.

In other embodiments, the cannabinoid concentrate of the present technology may be formulated together with one or more bioavailability-enhancing agents. Examples of bioavailability-enhancing agents that may be used include, but are not limited to: glycerol, vegetable, nut, or seed oils (such as coconut oil, peanut oil, soybean oil, safflower seed oil, corn oil, olive oil, castor oil, cottonseed oil, arachis oil, sunflower seed oil, coconut oil, palm oil, rapeseed oil, evening primrose oil, grape seed oil, wheat germ oil, sesame oil, avocado oil, almond, borage, peppermint and apricot kernel oils) and animal oils (such as fish liver oil, shark oil and mink oil). Further examples of bioavailability-enhancing agent that may be used include: polypeptides (such as gelatin, casein, and caseinate), polysaccharides (such as starch, dextrin, dextran, pectin, and gum arabic), as well as whole milk, skimmed milk, milk powder or mixtures of these. However, it is also possible to use polyvinyl alcohol, vinyl polymers, for example polyvinylpyrrolidone, (meth)acrylic acid polymers and copolymers, methylcellulose, carboxymethylcellulose, hydroxypropylcellulose and alginates.

In other embodiments, the cannabinoid concentrate of the present technology may comprise at least one carrier oil. The carrier oil may be used to reduce the viscosity of the cannabis concentrate before adding to the cocoa butter. Further, in the case of solid cannabis isolate (e.g., crystalline CBD), the carrier oil aids in its dissolution. Particularly suitable carrier oils include natural oils as known in the art, for example, edible vegetable oils. In some alternative embodiments, the carrier oils can include synthetic edible oils, for example, hydrogenated vegetable oils, medium chain triglyceride (MCT) oils, and the like and combinations thereof.

A non-limiting list of such exemplary carrier oils includes medium-chain triglycerides (MCT oil), medium-chain fatty acids (e.g., caproic acid, caprylic acid, capric acid, lauric acid), long-chain triglycerides (LCT oil), long chain fatty acids (e.g., myristic acid, palmitic acid, stearic acid, arachidic acid, linoleic acid), glycerine/glycerol, maisine cc, glycerol monolinoleate, coconut oil, corn oil, canola oil, olive oil, avocado oil, vegetable oil, flaxseed oil, palm oil, palm kernel oil, peanut oil, sunflower oil, rice bran oil, safflower oil, jojoba oil, argan oil, grapeseed oil, castor oil, wheat germ oil, peppermint oil, hemp oil, sesame oil, terpenes, terpenoids, beta-myrcene, linalool, α-pinene, beta-pinene, beta-caryophyllene, caryophyllene oxide, α-humulene, nerolidol, D-limonene, L-limonene, para-cymene, eugenol, farnesol, geraniol, phytol, menthol, terpineol, α-terpineol, benzaldehyde, hexyl acetate, methyl salicylate, eucalyptol, ocimene, terpinolene, α-terpinene, isopulegol, guaiol, α-bisabolol and combinations thereof. Other suitable carrier oils include Labrasol, LabrafacLipophile WL 1349, Labrail M1944, Peceol, Plurol Oliqiue CC 497, Transcutol HP, Tween 80, Gelucire 48/16, and combinations thereof.

In an embodiment, the carrier oil is maisine cc.

In an embodiment, the carrier oil is MCT oil.

The weight ratio of the cannabis concentrate:carrier oil may be about 5:1 to about 1:5. In an embodiment, the weight ratio of the cannabis concentrate:carrier oil may be about 1:1.

In instances where a carrier oil is used, the carrier oil is mixed with the cannabis concentrate under heating between about 40° C. and about 50° C. to form a homogenous mixture.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1—Preparing Cannabinoid-Infused Cocoa Butter

Cannabinoid-infused cocoa butter was prepared in the following way. Briefly, cocoa beans were fermented and dried. Dried cocoa beans were roasted and then subjected to cracking and winnowing to remove the shells off the beans. The nibs were ground or crushed to liquefy the cocoa butter and to produce chocolate liquor or chocolate liquid. A roll refiner was then used to further reduce the particle size of the cocoa mass and to distribute the cocoa butter evenly throughout the mass. THC distillate was then mixed into the cocoa butter at a THC:cocoa butter ratio of 1:4 (20% THC/80% cocoa butter) at a temperature of 40° C. with mixing for 30 minutes. The resulting mixture was then placed in the conch machine with rollers to continuously knead the cocoa butter and to give rise to THC-infused cocoa butter.

### Example 2—Preparing Cannabinoid-Containing Chocolate

The THC-infused cocoa butter as prepared in Example 1 was mixed with non-cannabinoid-infused cocoa butter during the tempering stage of the chocolate preparation. Mixing was performed to ensure a homogenous distribution of the THC into the chocolate. The mixture was allowed to cool to form solid chocolate comprising THC.

### Example 3—Self-Emulsifying Cannabis Oil

A formulation for a self-emulsifying cannabis oil is as follows: Cannabinoid or cannabis resin: 1-90% (w/w); Surfactant: 5-75% (w/w); and Edible Oil Carrier: 5-75% (w/w).

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

### BIBLIOGRAPHY

Karami, Z., & Hamidi, M. (2016). Cubosomes: Remarkable drug delivery potential. Drug Discovery Today, 21(5), 789-801. http://dx.doi.org/10.1016/j.drudis.2016.01.004
Spicer, P. T. (2005). Progress in liquid crystalline dispersions: Cubosomes. Current Opinion in Colloid & Interface Science, 10(5-6), 274-279. doi: 10.1016/j.cocis.2005.09.004

1. A method of making cannabinoid-containing cocoa butter, the method comprising mixing cocoa butter with a cannabinoid concentrate during preparation of the cocoa butter.

2. The method according to claim 1, wherein the mixing of the cocoa butter with the cannabinoid concentrate involves infusing the cocoa butter with the cannabinoid concentrate.

3. The method according to claim 1 or 2, wherein the mixing is performed during conching of the cocoa butter.

4. The method according to claim 1 or 2, wherein the mixing is performed before conching of the cocoa butter.

5. The method according to claims 1 to 2, wherein the mixing is performed during refining of the cocoa butter.

6. The method according to claims 1 to 2, wherein the mixing is performed after grinding of the cocoa butter.

7. The method according to claim 1 or 2, wherein the mixing is performed after grinding but before conching of the cocoa butter.

8. The method according to any one of claims 1 to 7, wherein the cannabinoid concentrate comprises a cannabinoid distillate.

9. The method according to claim 8, wherein the cannabinoid distillate comprises a THC distillate.

10. The method according to claim 9, wherein the THC distillate is obtained from a THC-dominant Cannabis strain.

11. The method according to claim 9 or 10, wherein the THC distillate comprises between about 60% and about 90% THC.

12. The method according to any one of claims 1 to 7, wherein the cannabinoid concentrate comprises a CBD isolate.

13. The method according to any one of claims 1 to 7, wherein the cannabinoid concentrate comprises a mixture of THC distillate and CBD isolate.

14. The method according to claim 13, wherein the mixture of THC distillate and CBD isolate has a THC:CBD ratio of 1:1.

15. The method according to claim 8, wherein the cannabinoid distillate has a THC:CBD ratio of 4:3.

16. The method according to any one of claims 1 to 15, wherein the cannabinoid concentrate is mixed with the cocoa butter in a ratio of between about 5:95 to about 25:75.

17. The method according to any one of claims 1 to 16, wherein the cannabinoid concentrate is formulated into a delivery system.

18. The method according to any one of claims 1 to 17, wherein the cannabinoid concentrate is encapsulated.

19. The method according to claim 18, wherein the cannabinoid concentrate is encapsulated by at least one molecular encapsulation agent.

20. The method according to any one of claims 1 to 17, wherein the cannabinoid concentrate is incorporated into a self-emulsifying drug delivery system.

21. The method according to any one of claims 1 to 17, wherein the cannabinoid concentrate is formulated into coacervates.

22. The method according to any one of claims 1 to 17, wherein the cannabinoid concentrate is formulated into cubosomes.

23. The method according to any one of claims 1 to 22, wherein the cannabinoid concentrate is edible.

24. A method of making a cocoa butter-derived product comprising cannabinoid, the method comprising:
i) mixing cocoa butter with a cannabinoid concentrate to obtain a cannabinoid-containing cocoa butter; and
ii) formulating the cannabinoid-containing cocoa butter to the cocoa butter-derived product.

25. The method according to claim 24, wherein the cocoa butter-derived product is chocolate.

26. The method according to claim 24, wherein the step of mixing of the cocoa butter with the cannabinoid concentrate involves infusing the cocoa butter with the cannabinoid concentrate.

27. The method according to claim 26, wherein the cannabinoid-containing cocoa butter is cannabinoid-infused cocoa butter.

28. The method according to any one of claims 24 to 27, wherein the cannabinoid concentrate is mixed with conched cocoa butter.

29. The method according to any one of claims 24 to 27, wherein the cannabinoid concentrate is mixed with refined cocoa butter.

30. The method according to any one of claims 24 to 29, wherein the cannabinoid concentrate comprises a cannabinoid distillate.

31. The method according to claim 30, wherein the cannabinoid distillate comprises a THC distillate.

32. The method according to claim 31, wherein the THC distillate is obtained from a THC-dominant Cannabis strain.

33. The method according to claim 31 or 32, wherein the THC distillate comprises between about 60% and about 90% THC.

34. The method according to any one of claims 24 to 29, wherein the cannabinoid concentrate comprises a CBD isolate.

35. The method according to any one of claims 24 to 29, wherein the cannabinoid concentrate comprises a mixture of THC distillate and CBD isolate.

36. The method according to claim 35, wherein the mixture of THC distillate and CBD isolate has a THC:CBD ratio of 1:1.

37. The method according to claim 30, wherein the cannabinoid distillate has a THC:CBD ratio of 4:3.

38. The method according to any one of claims 24 to 37 wherein the cannabinoid concentrate is mixed with the cocoa butter in a ratio of between about 5:95 to about 25:75.

39. The method according to any one of claims 24 to 38, wherein the cannabinoid concentrate is formulated into a delivery system.

40. The method according to any one of claims 24 to 38, wherein the cannabinoid concentrate is encapsulated.

41. The method according to any one of claims 1 to 40, wherein the cannabinoid concentrate comprises a carrier oil.

42. The method according to claim 41, wherein the carrier oil includes medium-chain triglycerides, medium-chain fatty acids, long-chain triglycerides, long chain fatty acids, glycerol or any combination thereof.

43. A cocoa butter-derived product comprising cannabinoid, the cocoa butter-derived product comprising the cannabinoid-containing cocoa butter obtained by the method as defined in any one of claims 1 to 42.

44. A cocoa butter-derived product comprising;
i) a cannabinoid; and
ii) cocoa butter;
the cocoa butter being infused with the cannabinoid in a cocoa butter:cannabinoid ratio of between about 95:5 and about 5:95.

45. The cocoa butter-derived product according to claim 44, the cocoa butter being infused with the cannabinoid in a cocoa butter:cannabinoid ratio of between about 90:10 and about 80:20.

46. The cocoa butter-derived product according to claim 44, the cocoa butter being infused with the cannabinoid in a cocoa butter:cannabinoid ratio of between about 80:20 and about 67:33.

47. The cocoa butter-derived product according to claim 44, the cocoa butter being infused with the cannabinoid in a cocoa butter:cannabinoid ratio of between about 20:5 and about 75:25.

48. A cocoa butter-derived product comprising;
i) a cannabinoid; and
ii) cocoa butter;
the cannabinoid being infused in the cocoa butter in a cannabinoid:cocoa butter ratio of between about 5:95 and about 95:5.

49. The cocoa butter-derived product according to claim 48, the cannabinoid being infused in the cocoa butter in a cannabinoid:cocoa butter ratio of between about 10:90 and about 20:80.

50. The cocoa butter-derived product according to any one of claims 44 to 49, wherein the cannabinoid is a cannabinoid concentrate.
